(19) United States
(12) Patent Application Publication
(10) Pub. No.: US 2025/0302273 A1
(43) Pub. Date: Oct. 2, 2025

(54) MEDICAL SYSTEM, CONTROL DEVICE, CONTROL METHOD, AND CONTROL PROGRAM

(71) Applicants: OLYMPUS CORPORATION, Tokyo (JP); NATIONAL CANCER CENTER, Tokyo (JP)

(72) Inventors: Naoya HATAKEYAMA, Tokyo (JP); Masafumi HARAGUCHI, Tokyo (JP); Masaaki ITO, Tokyo (JP); Yuta SUZUKI, Tokyo (JP)

(73) Assignees: OLYMPUS CORPORATION, Tokyo (JP); NATIONAL CANCER CENTER, Tokyo (JP)

(21) Appl. No.: 19/049,566

(22) Filed: Feb. 10, 2025

(30) Foreign Application Priority Data
Mar. 28, 2024 (JP) 2024-052783

(51) Int. Cl. A61B 1/00

(52) U.S. Cl. CPC A61B 1/00006

(57) ABSTRACT

Provided is a control device for controlling movement of an endoscope. The endoscope acquires an image including a target of interest. The control device includes at least one processor. The processor is configured to acquire a position of the target of interest in a field of view of the endoscope, calculate a position of a dirt on the endoscope in the field of view, set a target region at a position different from the position of the dirt, and cause the endoscope to track the target of interest such that the target of interest is positioned within the target region based on the position of the target region and the position of the target of interest.

### CROSS-REFERENCE TO RELATED APPLICATIONS

This application claims the benefit of Japanese Patent Application No. 2024-52783, the content of which is incorporated herein by reference.

### Technical Field

The present invention relates to a medical system, a control device, a control method, and a control program.

### Background Art

In a known medical system in the related art, an endoscope acquires an image including a target object, and the endoscope is caused to track a target of interest, such as a treatment tool, by using a predetermined three-dimensional region set in the field of view of the endoscope (for example, refer to PTL 1). In another known technique, a treatment scene is detected, and the position of a predetermined three-dimensional region is offset, so that an endoscope is caused to track a target of interest while ensuring visibility of a peeling target site or the like (for example, refer to PTL 2). In another known technique, video information acquired before and after a tip lens of a surgical camera is rotated is analyzed to detect dirts adhered to the lens (for example, see PTL 3).

### Citation List

### Patent Literature

[PTL 1] PCT International Publication No. WO 2022/54428
[PTL 2] PCT International Publication No. WO 2022/54882
[PTL 3] Japanese Unexamined Patent Application, Publication No. 2013-197652

### Summary of Invention

One aspect of the present invention is a control device for controlling movement of an endoscope. The endoscope acquires an image including a target of interest. The control device includes at least one processor. The processor is configured to acquire a position of the target of interest in a field of view of the endoscope, calculate a position of a dirt on the endoscope in the field of view, set a target region at a position different from the position of the dirt, and cause the endoscope to track the target of interest such that the target of interest is positioned within the target region based on the position of the target region and the position of the target of interest.

Another aspect of the invention is a medical system including: an endoscope that acquires an image including a target of interest; a moving device including a robot arm and moving the endoscope; and a control device that controls the moving device based on a position of the target of interest in the image. The control device includes at least one processor. The processor is configured to acquire position information including a position of the target of interest in a field of view of the endoscope, calculate a position of a dirt on the endoscope in the field of view, set a target region at a position different from the position of the dirt, and cause the endoscope to track the target of interest such that the target of interest is positioned within the target region based on the position of the target region and the position of the target of interest.

Another aspect of the invention is a control method including: a processor acquiring a position of a target of interest in a field of view of an endoscope; the processor calculating a position of a dirt on the endoscope in the field of view; the processor setting a target region at a position different from the position of the dirt; and the processor causing the endoscope to track the target of interest such that the target of interest is positioned within the target region based on the position of the target region and the position of the target of interest.

Another aspect of the invention is a non-transitory computer-readable recording medium having a control program causing a processor to execute functions of: acquiring a position of a target of interest in a field of view of an endoscope; calculating a position of a dirt on the endoscope in the field of view; setting a target region at a position different from the position of the dirt; and causing the endoscope to track the target of interest such that the target of interest is positioned within the target region based on the position of the target region and the position of the target of interest.

### BRIEF DESCRIPTION OF THE DRAWINGS

FIG. 1 FIG. 1 is an external view of a medical system according to an embodiment of the present invention.

FIG. 2 FIG. 2 is a block diagram of the medical system in FIG. 1.

FIG. 3 FIG. 3 shows a three-dimensional target region set in a field of view of the endoscope.

FIG. 4 FIG. 4 is an endoscopic image showing an example of a cross section of the target region in FIG. 3.

FIG. 5A shows the size of the target region in the endoscopic image at a depth X1 in FIG. 3.

FIG. 5B shows the size of the target region in the endoscopic image at a depth X2 in FIG. 3.

FIG. 5C shows the size of the target region in the endoscopic image at a depth X3 in FIG. 3.

FIG. 6A shows an example in which the centroid position of two dirts that are close to each other is calculated as the position of the dirt.

FIG. 6B shows an example in which the centroid positions of two dirts that are close to each other are calculated as the positions of dirts.

FIG. 7A shows an example method for setting a new target region when there is one dirt in the field of view.

FIG. 7B shows an example method for setting a new target region when there are multiple dirts in the field of view.

FIG. 8 FIG. 8 is a flowchart of a control method executed by a control device of the medical system in FIG. 1.

FIG. 9A shows another example method for setting a new target region when there are multiple dirts in the field of view.

FIG. 9B shows still another example method for setting a new target region when there are multiple dirts in the field of view.

FIG. 10 FIG. 10 shows an example of movement of the endoscope when the field of view of the endoscope is set to an upward view.

FIG. 11A shows an example endoscopic image when the field of view of the endoscope is set to an upward view.

FIG. 11B shows an example endoscopic image when the field of view of the endoscope is set to a downward view.

FIG. 12A shows an example endoscopic image in the case where the field of view of the endoscope is set to an upward view, there is no dirt, and the target region is positioned at the center of the upper half.

FIG. 12B shows an example endoscopic image similar to that in FIG. 12A, but with a dirt adhered thereto.

FIG. 12C shows an example endoscopic image illustrating movement of the target region due to the dirt in FIG. 12B.

FIG. 12D shows an example endoscopic image when the endoscope is moved such that the tip of the treatment tool is positioned in the new target region in FIG. 12C.

FIG. 13A shows an example endoscopic image in the case where the field of view of the endoscope is set to the left half, there is no dirt, and the target region is positioned at the center of the left half.

FIG. 13B shows an example endoscopic image similar to that in FIG. 13A, but with a dirt adhered thereto.

FIG. 13C shows an example endoscopic image illustrating movement of the target region due to the dirt in FIG. 13B.

FIG. 13D shows an example endoscopic image when the endoscope is moved such that the tip of the treatment tool is positioned in the new target region in FIG. 13C.

FIG. 14 FIG. 14 shows an example endoscopic image with multiple candidate regions set in advance.

FIG. 15 FIG. 15 is a perspective view showing an example weight distribution in an endoscopic image, which is used for setting a new target region by means of a weight gradient given to dirts and the like in the same manner as in the potential gradient in the population potential method.

### DESCRIPTION OF EMBODIMENTS

A medical system 10, a control device 4, a control method, and a control program according to an embodiment of the present invention will be described with reference to the drawings.

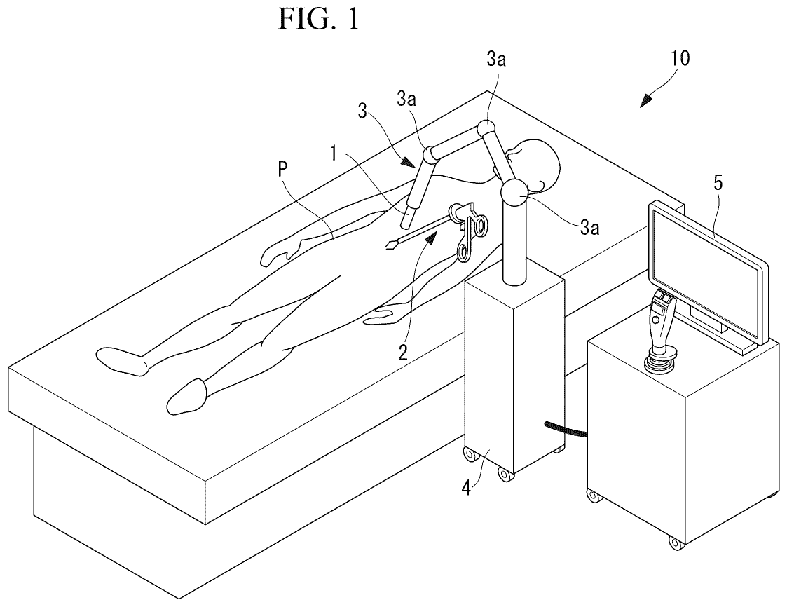

As shown in FIG. 1, the medical system 10 according to this embodiment includes: an endoscope 1 and a treatment tool 2 to be inserted into the body of a patient P; a moving device 3 that holds the endoscope 1 and moves the endoscope 1 in the body; the control device 4 that is connected to the endoscope 1 and the moving device 3 and controls the moving device 3; and a display device 5 for displaying an endoscopic image D.

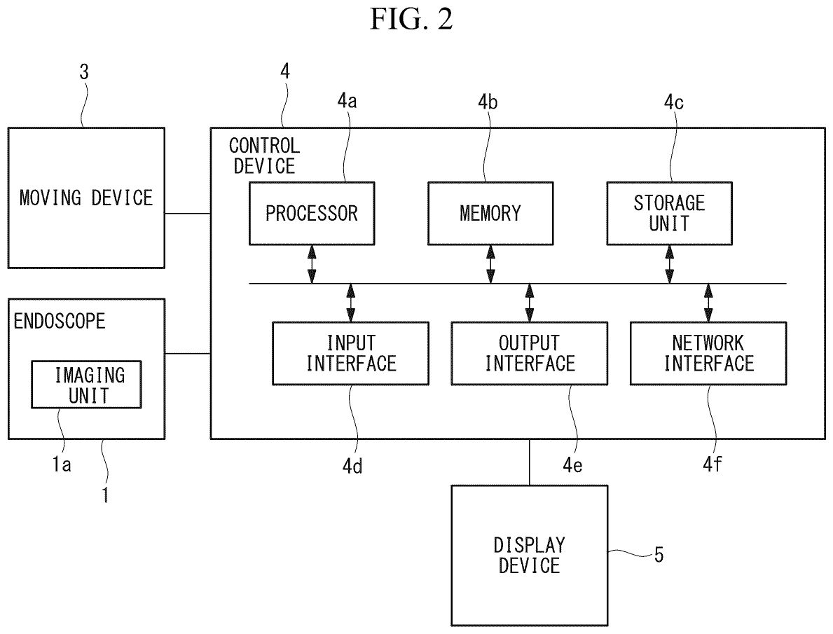

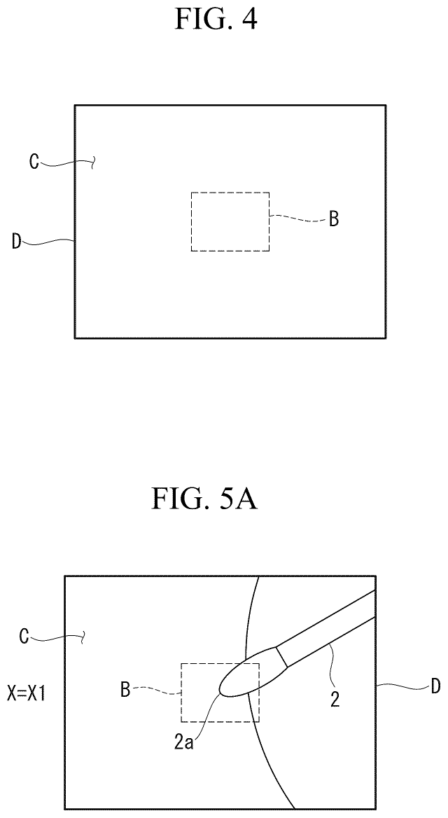

The endoscope 1 is, for example, a rigid endoscope having a tip lens, and includes an imaging unit 1a (see FIG. 2) that has an imaging device for acquiring an endoscopic image D (see FIG. 4). The endoscope 1 acquires an endoscopic image D including a tip 2a (hereinafter, also referred to as a target of interest) of the treatment tool 2 with the imaging unit 1a and transmits the endoscopic image D to the control device 4. The imaging unit 1a is, for example, a three-dimensional camera provided at the distal end of the endoscope 1 and acquires a stereo image including information about the three-dimensional position of the tip 2a of the treatment tool 2 as the endoscopic image D.

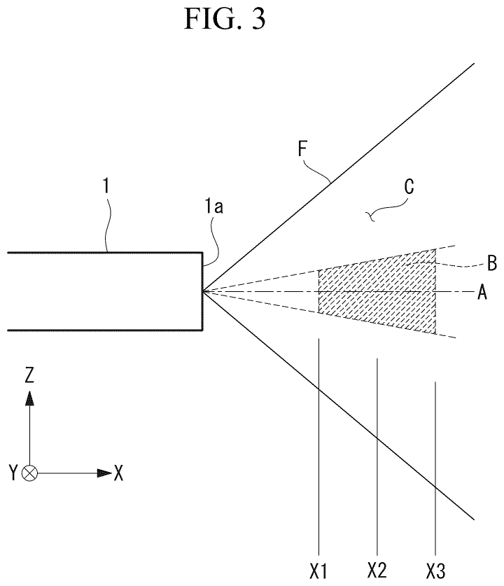

The moving device 3 is a robot arm having multiple joints 3a and holds the proximal end of the endoscope 1 at the tip of the robot arm. In one example, as shown in FIG. 3, the moving device 3 has three degrees of freedom of motion, namely: linear motion in the forward/backward direction along the X axis; rotation about the Y axis (pitch); and rotation about the Z axis (yaw). Preferably, the moving device 3 further has a degree of freedom of motion that is rotation about the X axis (roll). The X axis is an axis on the same straight line as the optical axis A of the endoscope 1. The Y axis and the Z axis are axes orthogonal to the optical axis A and extending in directions corresponding to the horizontal direction and the vertical direction of the endoscopic image D, respectively.

As shown in FIG. 2, the control device 4 includes at least one processor 4a, such as a central processing unit, a memory 4b, a storage unit 4c, an input interface 4d, an output interface 4e, and a network interface 4f.

The endoscopic images D transmitted from the endoscope 1 are sequentially input to the control device 4 via the input interface 4d, sequentially output to the display device 5 via the output interface 4e, and displayed on the display device 5. An operator, such as a surgeon, operates the treatment tool 2 inserted into the body while observing the endoscopic image D displayed on the display device 5, and performs treatment of an affected part in the body using the treatment tool 2.

The memory 4b includes a volatile storage device, such as a random access memory (RAM), and is used as a workspace for the processor 4a.

The storage unit 4c is a computer-readable non-transitory storage medium, and examples thereof include a known magnetic disc, optical disc, flash memory, and read-only memory (ROM).

The storage unit 4c stores programs and data necessary for causing the processor 4a to execute processing. Functions of the control device 4 described below are realized by the processor 4a reading a program into the memory 4b and executing the program. Some functions of the control device 4 may be realized by a dedicated logic circuit or the like.

The control device 4 has a manual mode and a tracking mode. In the manual mode, the operator, such as a surgeon, manually operates the endoscope 1, whereas, in the tracking mode, the control device 4 causes the endoscope 1 to automatically track the tip 2a of the treatment tool 2.

The control device 4 switches between the manual mode and the tracking mode according to an instruction from the operator. For example, the control device 4 includes artificial intelligence capable of recognizing a human voice. The control device 4 switches to the manual mode when recognizing a voice saying “manual mode”, and switches to the tracking mode when recognizing a voice saying “tracking mode”. The control device 4 may also switch between the manual mode and the tracking mode in accordance with ON/OFF of a manual operation switch (not shown) provided on the endoscope 1.

In the manual mode, for example, the operator, such as a surgeon, can remotely operate the moving device 3 by operating an operation device (not shown) connected to the control device 4.

In the tracking mode, the control device 4 controls the moving device 3 on the basis of the three-dimensional position of the tip 2a of the treatment tool 2 to cause the endoscope 1 to three-dimensionally track the tip 2a of the treatment tool 2 such that the tip 2a of the treatment tool 2 moves toward the center of a target region B.

Specifically, the control device 4 recognizes the treatment tool 2 in the endoscopic image D and calculates the three-dimensional position of the tip 2a of the treatment tool 2 using the endoscopic image D. Then, the control device 4 operates the joints 3a such that the optical axis A of the endoscope 1 moves in a direction intersecting the optical axis A, toward the tip 2a of the treatment tool 2, and such that the tip of the endoscope 1 moves in the depth direction along the optical axis A, toward a position separated from the tip 2a by a predetermined observation distance.

The target region B is a predetermined three-dimensional region set in the field of view F of the endoscope 1 and has dimensions in the X direction, the Y direction, and the Z direction orthogonal to one another. The X direction is a depth direction parallel to the optical axis A of the endoscope 1. The Y direction and the Z direction are directions orthogonal to the optical axis A and parallel to the horizontal direction and the vertical direction of the endoscopic image D, respectively.

As shown in FIGS. 3 and 4, the target region B is positioned at a position separated from the tip of the endoscope 1 in the X direction and is set in an area of the field of view F in the X direction. Furthermore, the target region B has such a three-dimensional shape that the cross section thereof at a position closer to the tip of the endoscope 1 is smaller. In an initial state in which the target region B is not specifically designated, the target region B in the endoscopic image D is a region at the central portion, including the center, of the endoscopic image D. the cross-sectional shape of the target region B orthogonal to the optical axis A may be any of a rectangle, a circle, and an ellipse. Another shape, such as a polygon, is also possible. The target region B may be superimposed on the endoscopic image D, or may not be displayed.

In one example, the cross-sectional shape of the target region B is a shape similar to the shape of the endoscopic image D. For example, when the endoscopic image D is rectangular, the cross section of the target region B is also rectangular. The target region B displayed over the endoscopic image D may hinder the observation of the endoscopic image D, so, preferably, the target region B is not displayed. When the target region B has a shape similar to the shape of the endoscopic image D, the surgeon can easily recognize the position of the target region B that is not displayed.

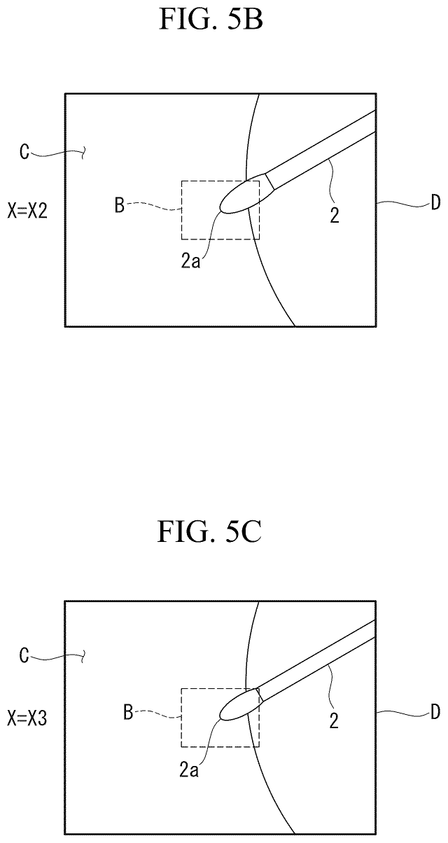

Typically, the field of view F of the endoscope 1 has a cone shape with a vertex at the tip or in the vicinity of the tip of the endoscope 1. Preferably, the target region B has a frustum shape having a common vertex with the field of view F of the endoscope 1. With this target region B, as shown in FIGS. 5A to 5C, the apparent size and position of the target region B in the endoscopic image D are constant regardless of the positions X1, X2, and X3 in the X direction.

The processor 4a processes the endoscopic image D transmitted from the endoscope 1, detects a dirt adhered to the tip lens of the endoscope 1 by using a known method, and calculates the position of the detected dirt in the YZ direction, the position of the dirt is a pixel position (coordinates) in the endoscopic image D where the detected dirt is positioned, and is calculated according to the dirt detection rule below, for example.

### Dirt Detection Rule

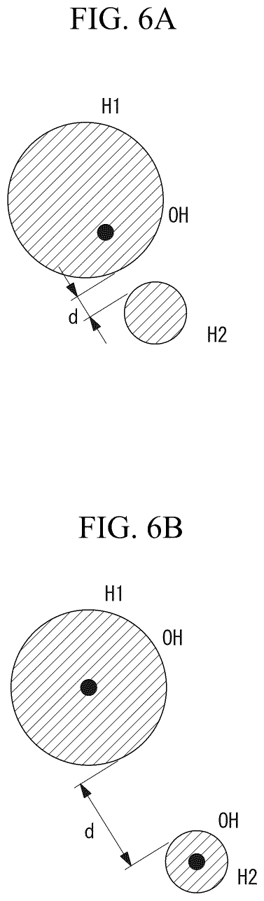

(a) When a dirt exists on a single pixel or on multiple consecutive pixel areas, and when the number of pixels (area) is smaller than or equal to a first threshold, it is regarded that there is no dirt.
(b) When a dirt exists on multiple consecutive pixel areas, and when the number of pixels is larger than the first threshold, the position of the dirt is the centroid pixel position of the multiple pixels.
(c) As shown in FIG. 6A, when dirts exist on multiple non-consecutive pixel areas, and when the distance d between dirt areas H1 and H2 is smaller than or equal to a second threshold, the dirt areas are regarded as a single dirt area. Then, the pixel position corresponding to the centroid position OH (representative value) determined by the ratio of the areas of the dirt areas H1 and H2 is regarded as the dirt position. In the drawings, the dirts are illustrated as circles for the sake of simplicity, but the dirts to be detected may have any shape.
(d) As shown in FIG. 6B, when dirts exist on multiple non-consecutive pixel areas, and when the distance d between the dirt areas H1 and H2 is larger than the second threshold, the pixel positions corresponding to the centroid positions OH of the dirt areas H1 and H2 are regarded as the positions of the dirts.

The processor 4a calculates the position of the tip 2a of the treatment tool 2 on the basis of the endoscopic image D transmitted from the endoscope 1. Furthermore, the processor 4a sets the position of a new target region B on the basis of the calculated position OH of the dirt.

The position of the new target region B is set in accordance with the target position setting rule described below, for example.

### Target Position Setting Rule

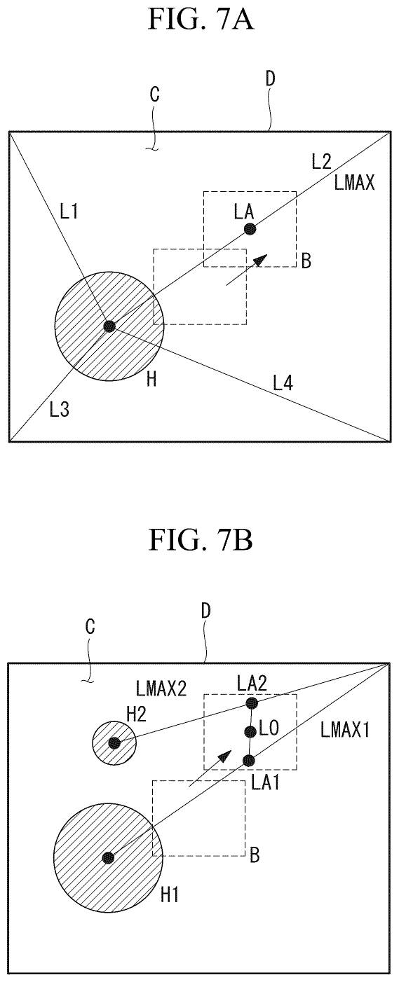

(e) When only a single dirt area OH is detected, as shown in FIG. 7A, the processor 4a calculates the lengths of four straight lines L1 to L4 connecting the position of the dirt, which is the centroid position OH of the dirt area H, and the four corners (ends of the field of view) of the endoscopic image D. Then, the processor 4a sets the center position LA of the longest straight line (here, L2) among the four straight lines L1 to L4 as the position (center position) of the new target region B.
(f) When multiple dirt areas H are detected, as shown in FIG. 7B, the processor 4a calculates the center positions LA1 and LA2 of the longest straight lines LMAX and LMAX2 for the dirt areas H1 and H2, respectively, in the same manner as described above. Then, the processor 4a sets the median value LO of the center positions LA1 and LA2 of all the longest straight lines LMAX and LMAX2 as the position of the new target region B.

The processor 4a calculates the amounts by which the joints 3a of the moving device 3 are to be driven on the basis of the set position of the target region B and the calculated position of the tip 2a of the treatment tool 2, and operates the moving device 3. In this way, the target region B is set at a position different from the positions of the dirts H, H1, and H2, and the endoscope 1 is moved to track the treatment tool 2 such that the detected tip 2a of the treatment tool 2 is positioned in the set target region B.

Next, the operation of the medical system 10 according to this embodiment will be described below with reference to the drawings.

The surgeon performs treatment by operating the treatment tool 2 inserted into the body of the patient P while observing the endoscopic image D displayed on the display device 5. During the treatment, the operator, such as the surgeon, switches from the manual mode to the tracking mode or from the tracking mode to the manual mode by, for example, voice.

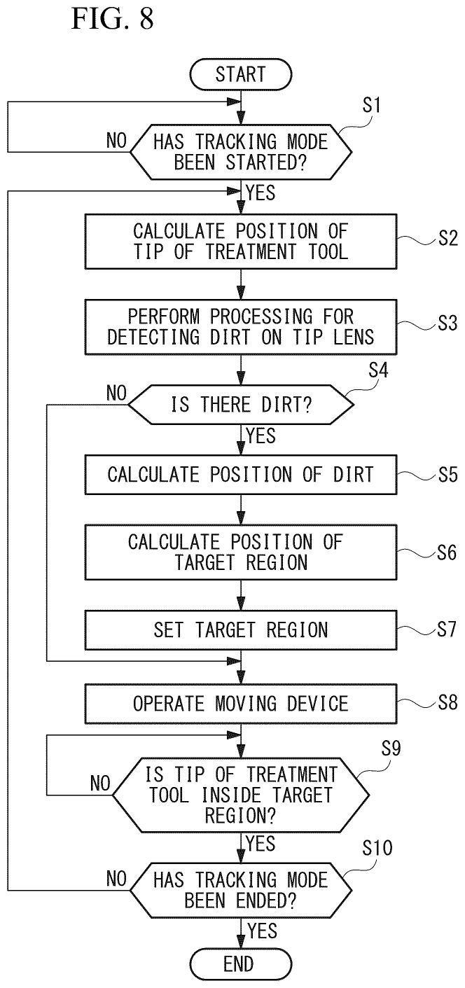

As shown in FIG. 8, when the mode is switched to the tracking mode in step S1, the processor executes the control method including steps S2 to S10 to control the moving device 3 in the tracking mode.

In the control method according to this embodiment, first, the processor 4a calculates the position of the tip 2a of the treatment tool on the basis of the endoscopic image D (step S2). Next, the processor 4a performs processing for detecting a dirt on the tip lens of the endoscope 1 on the basis of the endoscopic image D (step S3) to determine the presence/absence of a dirt (step S4). When it is determined that there is a dirt on the tip lens, the processor 4a calculates the position of the dirt (step S5).

Next, the processor 4a calculates the position of a new target region B in accordance with the target position setting rule described above on the basis of the position of the dirt (step S6), and sets the target region B at the calculated position (step S7). Then, the moving device 3 is operated such that the tip 2a of the treatment tool 2 is positioned in the set target region B (step S8). It is determined whether or not the tip 2a of the treatment tool 2 is positioned in the new target region B (step S9), and when the tip 2a is not positioned in the new target region B, the process is repeated from step S8.

Then, it is determined whether or not the operator, such as the surgeon, has switched modes from the tracking mode to the manual mode by voice, for example (step S10). If switching has not been performed, the process is repeated from step S2.

As described above, according to this embodiment, the new target region B is set in a dirt-free region in the field of view. Hence, even if a dirt adheres to the tip lens, it is possible to capture the tip 2a of the treatment tool 2 while avoiding the dirt. This provides an advantage in that it is possible to ensure, around the tip 2a of the treatment tool 2, a wide field of view that is not blocked by a dirt, allowing the surgeon to easily continue treatment.

Furthermore, according to this embodiment, when a dirt exists on a single pixel or on multiple consecutive pixel areas, and when the number of pixels is smaller than or equal to the first threshold, it is regarded that there is no dirt. This improves the ease of treatment by preventing unnecessary processing of moving the target region B due to a small dirt that does not hinder the field of view, and preventing unnecessary movement of the target region B.

Furthermore, when a dirt exists on multiple consecutive pixel areas, and when the number of pixels is larger than the first threshold, the position of the dirt is regarded as the centroid pixel position of the multiple pixels. Hence, a relatively large mass of dirts can be processed as a single dirt. Furthermore, because multiple dirts that are close to each other are regarded as a single dirt, the processing can be simplified.

According to this embodiment, the center position of the new target region B is set to the center position LA of the longest straight line LMAX among the straight lines L1 to L4 connecting the dirt detected in the field of view and the four corners of the endoscopic image D. Furthermore, when multiple dirt areas are detected, the median value LO of the center positions LA1 and LA2 of the longest straight lines MAX1 and LMAX2 for the dirt areas is set as the position of the new target region B. In this way, the new target region B can be positioned in a dirt-free region within the field of view by a simple method.

In this embodiment, the center position of the new target region B is set at the center point of the longest straight line LMAX among the straight lines connecting the dirt detected in the field of view and the four corners of the endoscopic image D. Instead, the new target region B may be set at another position on the straight line LMAX that is not the center point of the straight line LMAX. Furthermore, the new target region B may be set not only on the longest straight line LMAX, but also on any straight line among straight lines having a predetermined length or more.

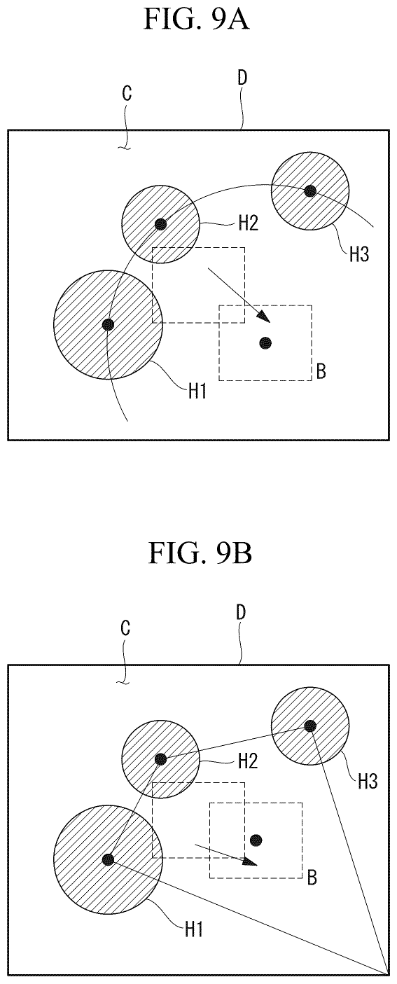

Furthermore, as shown in FIG. 9A, when there are multiple dirts H1, H2, and H3 that are relatively distant from one another, the center position of the new target region B may be set at the center position of a circle passing through the positions of the dirts H1, H2, and H3. The target region B may be set at the center of an ellipse or, as shown in FIG. 9B, a polygon including any corner of the endoscopic image D, instead of a circle. In this way, a new target region B can be set in a relatively wide region where the field of view is not blocked by a dirt, and the operator can continue treatment while observing the tip 2a of the treatment tool 2 in a wider field of view. Although an example case where there are dirts H1, H2, and H3 has been described, the number of dirts is not limited to three. As long as there is more than one dirt, the target region B can be set using this method regardless of the number of the dirts.

In addition, in order to prevent the new target region B from being set outside the endoscopic image D, the allowable coordinates of the center position of the new target region B to be calculated may be limited within a predetermined range at the center of the endoscopic image D.

Furthermore, as shown in FIG. 3, because the field of view F of the endoscope 1 has a cone shape having a vertex at the tip or in the vicinity of the tip of the endoscope 1, the upper half of the field of view F is an upward view, in which an observation target such as a tissue to be treated is viewed from below, and the lower half of the field of view F is a downward view, in which the observation target is viewed from above. Which of the upward view and the downward view is desired depends on the type of the treatment to be performed.

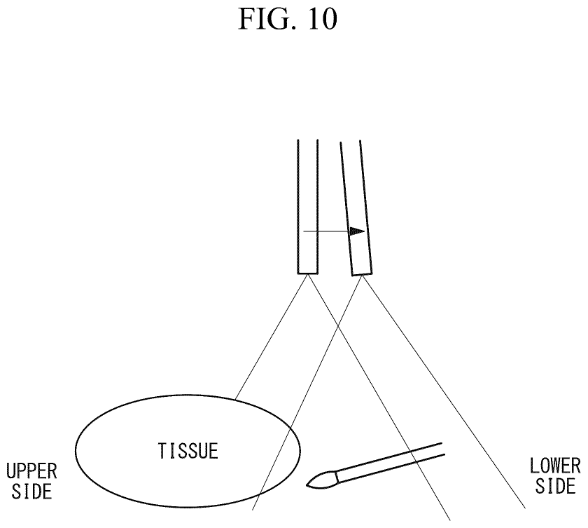

Specifically, when an operator, such as a surgeon, wishes to perform treatment in an upward view, the processor 4a operates, according to an instruction from the operator, the moving device 3 to move the endoscope 1 such that the observation target is captured on the upper side in the field of view, as shown in FIG. 10. As shown in FIG. 10, by setting to the upward view, it is possible to observe the treatment tool 2 without being blocked by the tissue even when the treatment tool 2 is hidden by the tissue when the target region is positioned at the center of the field of view.

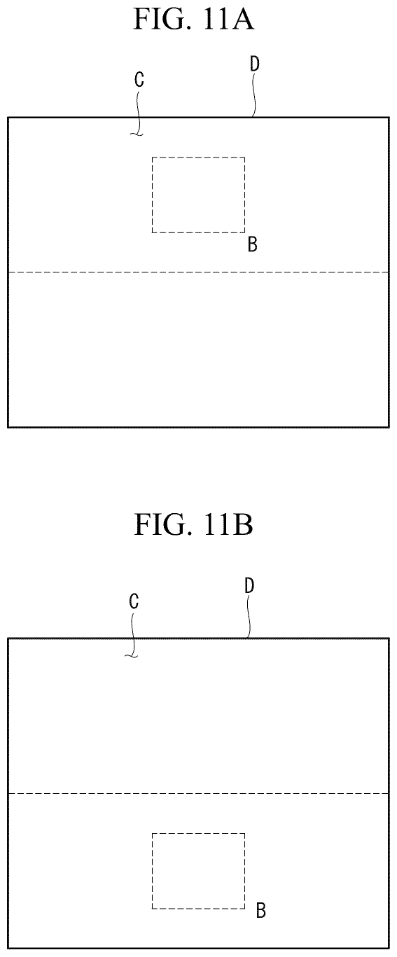

In contrast, when the operator, such as a surgeon, wishes to perform treatment in a downward view, the processor 4a operates, according to an instruction from the operator, the moving device 3 to move the endoscope 1 such that the observation target is captured on the lower side in the field of view. In this way, as shown in FIG. 11A, the target region B is positioned at the center of the upper half of the field of view F, whereas, when a downward view is desired, for example, as shown in FIG. 11B, the target region B is positioned at the center of the lower half of the field of view F.

In these cases, when the target region B is moved in response to detection of a dirt, it is preferable that the upward view or the downward view be maintained in the new target region B. Hence, to cope with these cases, for example, a settable region that can be set as the target region B may be stored in the memory 4b, and the processor 4a may set a new target region B within the settable region.

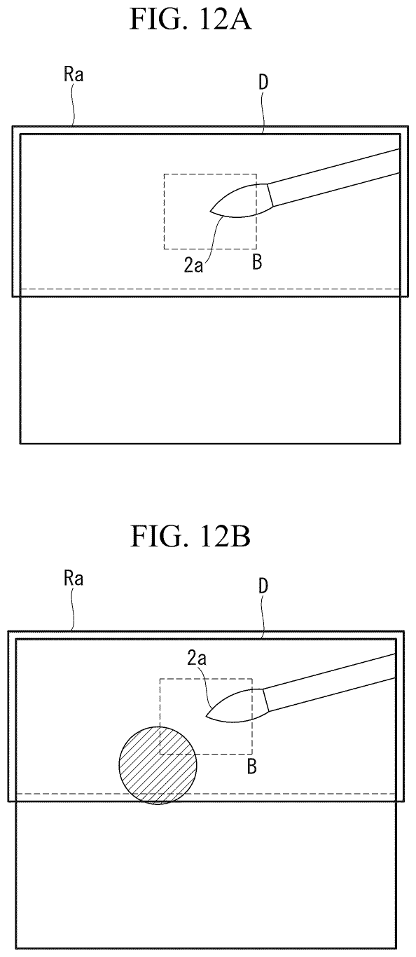

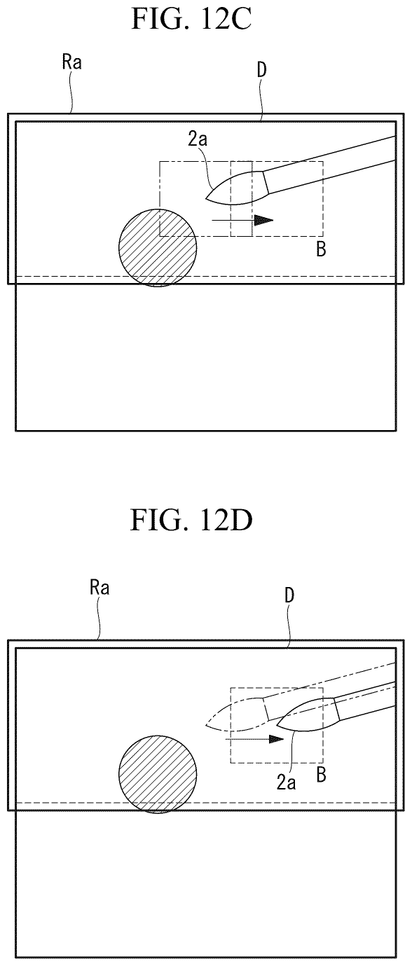

That is, when treatment is to be performed with the upward view, as shown in FIG. 12A, the upper half of the field of view is stored in the memory 4b as the settable region, and a new target region B is limited to the inside of the upper half of the field of view. In this state, when a dirt is detected as shown in FIG. 12B, the target region B is set at a position shifted mainly horizontally (to the right side in FIG. 12C) in the upper half of the field of view, as shown in FIG. 12C. Then, as shown in FIG. 12D, the endoscope 1 is moved so that the tip 2a of the treatment tool 2 is positioned in the newly set target region B.

Similarly, when the treatment is to be performed with the downward view, the lower half of the field of view is stored as the settable region in the memory, and the new target region B is limited to the inside of the lower half of the field of view. Hence, the target region B is set to move mainly horizontally in the lower half of the field of view.

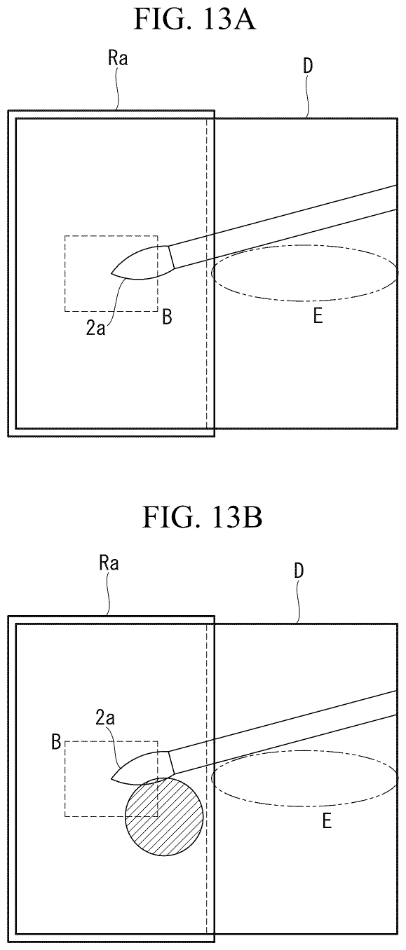

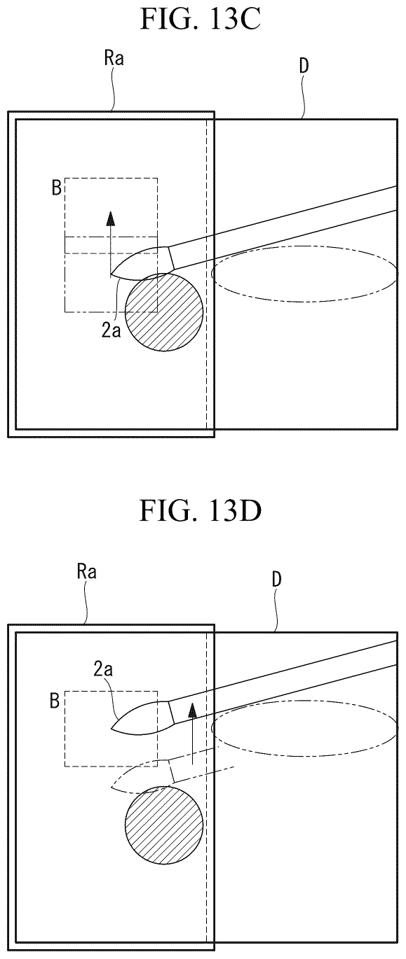

Furthermore, as shown in FIG. 13A, when a treatment is performed in which a tissue is incised with the treatment tool being moved from the left side to the right side in the endoscopic image D, it is required to ensure a wide field of view at an incision destination (region E) in front of (in FIG. 13A, to the right of) the treatment tool 2 in the movement direction thereof. In that case, the processor 4a operates the moving device 3 to move the endoscope 1 such that the tip 2a of the treatment tool 2 is captured on the left side of the field of view in response to the instruction of the operator, and the target region B is positioned at the center of the left half of the field of view.

Hence, when the target region B is moved in response to detection of a dirt, the left half of the field of view is stored as a settable region in the memory 4b, and a new target region B is also set within the left half of the field of view so as to move mainly vertically. Specifically, when a dirt is detected as shown in FIG. 13B, the target region B is set at a position shifted mainly vertically (the upper side in FIG. 13C) in the left half of the field of view, as shown in FIG. 13C. Then, as shown in FIG. 13D, the endoscope 1 is moved so that the tip 2a of the treatment tool 2 is positioned in the newly set target region B.

Similarly, in a treatment in which it is required to ensure a wide field of view on the left side in the field of view, the right half of the field of view is stored as a settable region in the memory 4b, and a new target region B is also set so as to move mainly vertically within the right half of the field of view.

In this way, even if a dirt adheres to the tip lens, it is possible to capture the tip 2a of the treatment tool 2 while avoiding the dirt, under the same conditions of the field of view.

The same effect can be obtained by limiting the direction in which the target region B is moved in response to detection of a dirt, instead of storing the settable region and limiting the new target region B to the inside of the settable region. The settable region is not limited to the upper half, the lower half, the left half, or the right half of the field of view, and may be set in any one of three or more fields of view obtained by horizontally or vertically dividing the field of view.

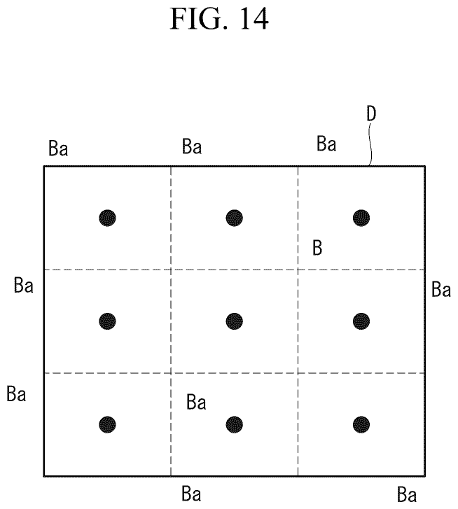

Furthermore, instead of storing the settable region, multiple candidate regions Ba that can be set as the target region B in the field of view may be stored in the memory 4b in advance, and the processor 4a may set any of the candidate regions Ba that do not include a dirt as a new target region B. In the example shown in FIG. 14, the center positions of the candidate regions Ba are set at a total of nine equally spaced positions, i.e., three horizontal positions and three vertical positions in the field of view, as indicated by nine black circles.

Because a new target region B is selected from the candidate regions Ba stored in advance, it is possible to prevent a part of the target region B from protruding outside the endoscopic image D, and thus to maintain the ease of observation. Although the example in which the candidate regions Ba do not overlap one another has been shown, the candidate regions Ba may overlap one another or may be spaced apart from one another.

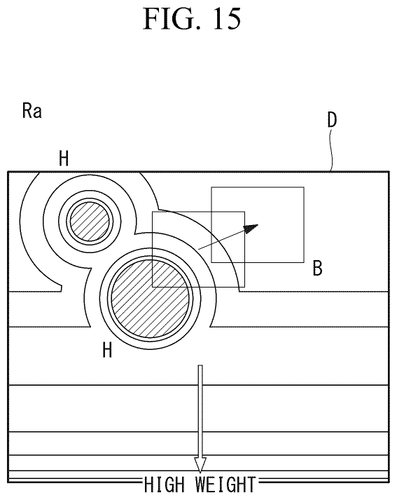

Furthermore, similarly to the potential gradient in the population potential method, a weight may be given to each position in the endoscopic image D, and a weight gradient may be set such that the smaller the weight, the further away from the dirt. Then, a new target region may be set to a region having the smallest weight. For example, as shown in FIG. 15, by giving a weight such that the weight is highest at the calculated position of the dirt and decreases toward the periphery thereof, it is possible to set a weight gradient that decreases as the distance from the dirt increases. The thin lines in FIG. 15 are the contour lines of the weight. Furthermore, for example, in the case of the upward view described above, by setting a weight gradient that becomes lower toward the upper side in the lower half of the field of view, it is possible to prevent a new target region from being set in the lower half of the field of view, with the same mechanism as that of the dirt.

Furthermore, the target region B may be reset to the center of the field of view at the timing when the endoscope 1 is removed from the body of the patient P or the removed endoscope 1 is inserted into the body of the patient P again after the target region B has been moved. When the field of view has been offset, as in the upward view, the downward view, or a horizontally wide angle view, immediately before the endoscope 1 is removed from the body of the patient P, the position of the target region B to be reset may be returned to the previously offset direction.

Although the endoscope 1 acquires a three-dimensional stereo image as the endoscopic image D in the above-described embodiment, the endoscope 1 may acquire a two-dimensional endoscopic image D instead. In that case, for example, the position of the tip 2a of the treatment tool 2 in the X direction may be measured with another ranging means, such as a range sensor provided at the tip of the endoscope 1.

In the embodiment described above, the target of interest to be tracked by the endoscope 1 is the treatment tool 2. However, the target of interest is not limited thereto, and may be any object appearing in the endoscopic image D during surgery. For example, the target of interest may be a lesion, an organ, a blood vessel, a medical material such as a marker or gauze, or a medical instrument other than the treatment tool 2.

Although the embodiment of the present invention and the modifications thereof have been described in detail with reference to the drawings, the specific configuration of the present invention is not limited to the above-described embodiments and modifications, and various design changes can be made without departing from the scope of the present invention. The components described in the above embodiments and modifications may be combined as appropriate.

For example, an object may be a lumen other than the large intestine, or may be an organ other than the lumen that can be a target of the endoscopic examination. The region of interest may be set according to the object.

### REFERENCE SIGNS LIST

1 Endoscope
2 Treatment tool
2a Tip (target of interest)
3 Moving device
4 Control device
4a Processor
4b Memory
10 Medical system
D Endoscopic image (image)

1. A control device for controlling movement of an endoscope, the endoscope acquiring an image including a target of interest, the control device comprising:
at least one processor, wherein the processor is configured to:
acquire a position of the target of interest in a field of view of the endoscope;
calculate a position of a dirt on the endoscope in the field of view;
set a target region at a position different from the position of the dirt; and
cause the endoscope to track the target of interest such that the target of interest is positioned within the target region based on the position of the target region and the position of the target of interest.

2. The control device according to claim 1, wherein the processor is configured to set a center of the target region on a straight line connecting the position of the dirt and an end of the field of view.

3. The control device according to claim 2, wherein the end of the field of view is an end farthest from the position of the dirt.

4. The control device according to claim 1, wherein, when a distance between a plurality of dirts present in the field of view is smaller than or equal to a predetermined threshold, the processor is configured to calculate a representative position of the plurality of dirts as the position of the dirt, and, when the distance between the plurality of dirts present in the field of view is larger than the threshold, the processor is configured to calculate each of positions of the plurality of dirts as the position of the dirt.

5. The control device according to claim 4, wherein, when the processor is configured to calculate each of the positions of the plurality of dirts, the processor is configured to set a center of the target region to a median value of centers of multiple straight lines connecting each of the positions of the plurality of dirts and the end of the field of view.

6. The control device according to claim 4, wherein the processor is configured to set a center of the target region at a centroid position of a polygon, a circle, or an ellipse passing through the positions of the plurality of dirts.

7. The control device according to claim 1, wherein the processor is configured to calculate an area of the dirt, and calculate the position of the dirt only when the area of the dirt is larger than a predetermined threshold.

8. The control device according to claim 1, further comprising at least one memory, wherein
the memory stores a settable region that can be set as the target region in the field of view, and
the processor is configured to set the target region in the settable region.

9. The control device according to claim 1, further comprising at least one memory, wherein
the memory stores multiple candidate regions that can be set as the target region in the field of view, and
the processor is configured to set the target region in any of the candidate regions that do not include the calculated position of the dirt.

10. The control device according to claim 1,
wherein the processor is configured to generate a weight gradient by giving a weight that decreases from the calculated position of the dirt toward a periphery in the field of view, and set a center of the target region at a position where the weight is lowest.

11. A control method comprising:
a processor acquiring a position of a target of interest in a field of view of an endoscope;
the processor calculating a position of a dirt on the endoscope in the field of view;
the processor setting a target region at a position different from the position of the dirt; and
the processor causing the endoscope to track the target of interest such that the target of interest is positioned within the target region based on the position of the target region and the position of the target of interest.

12. A non-transitory computer-readable recording medium having a control program causing a processor to execute functions of:
acquiring a position of a target of interest in a field of view of an endoscope;
calculating a position of a dirt on the endoscope in the field of view;
setting a target region at a position different from the position of the dirt; and
causing the endoscope to track the target of interest such that the target of interest is positioned within the target region based on the position of the target region and the position of the target of interest.
